(19) United States
(12) Patent Application Publication
(10) Pub. No.: US 2024/0325598 A1
(43) Pub. Date: Oct. 3, 2024

(54) COMPOSITIONS HAVING CHAIR-SIDE HANDLING PROPERTIES FOR DENTAL AND SOFT TISSUE APPLICATIONS

(71) Applicant: EVONIK OPERATIONS GMBH, Essen (DE)

(72) Inventors: Jian-Feng Zhang, Vestavia, AL (US); Donghui Wang, Hoover, AL (US); Balaji Prabhu, Hoover, AL (US); Donghun Koo, Hoover, AL (US); Marshall Scott Jones, Bessemer, AL (US); Andrea Engel, Hoover, AL (US); Mahrokh Dadsetan, Hoover, AL (US); Stefan Randl, Frankfurt (DE); Thiago Francisco Costa Carpes Borges, Sterrett, AL (US)

(73) Assignee: EVONIK OPERATIONS GMBH, Essen (DE)

(21) Appl. No.: 18/708,527

(22) PCT Filed: Nov. 2, 2022
(86) PCT No.: PCT/EP2022/080515

§ 371 Date: May 8, 2024

Related U.S. Application Data
(60) Provisional application No. 63/278,031, filed on Nov. 10, 2021.

(51) Int. Cl. A61L 27/18; A61L 27/26; A61L 27/54

(52) U.S. Cl. CPC A61L 27/18; A61L 27/26; A61L 27/54; A61L 2430/12

(57) ABSTRACT

The present invention is directed to compositions having chair-side handling properties comprising polymers for dental, soft tissue and combination products applications.

### FIELD OF THE INVENTION

This invention relates to compositions having chair-side handling properties comprising biodegradable polymers for dental, soft tissue and combination products applications.

### BACKGROUND OF THE INVENTION

Biodegradable polymers have received increasing demand for medical implants with an advantage that eliminates the needs of surgical removal. These polymers are poly(L-lactide) (PLLA), poly(D,L-lactide) (PDLLA), polyglycolide (PGA), poly(lactide-co-glycolide) (PLGA), poly(L-lactide-co-D,L-lactide) (PLLA-co-PDLLA). These polymers are rigid, hard and brittle around room or body temperature, which is normally below their glass transition temperatures (ca. 35-65° C.). Hence, they are difficult to further manipulate the shape at room or body temperature to better fit the implant site. Although polydioxanone (PDO) and polycaprolactone (PCL) have been considered flexible (i.e., low modulus) biodegradable polymers. However, they are unable to hold at a new shape at room or body temperature if they are not restricted.

In certain applications, for example in oral surgery, and particularly in the field of oral guided tissue such as periodontal ligament regeneration (GTR) and guided bone regeneration (GBR), a dental membrane is typically involved. GTR and GBR are surgical techniques performed to regenerate the tooth supporting tissues (GTR) and the alveolar bone in edentulous areas (GBR), respectively. GTR is often used together with GBR in areas focusing on regrowing bone where the GBR prevents the connective soft tissue from ingrowing into the space priority of bone tissue. Such a functionalized membrane integrating promotion of soft tissue and hard tissue which normally requires incorporation of osteoconductive fillers, such as hydroxyapatite, is beneficial. However, incorporation of biologically active agents/additives for example animal derived collagen to synthetic biodegradable polymers to improve bioactivity remains a challenge. Majority of polymers require high temperature to melt during thermal processing yet this temperature is beyond the tolerance for certain biologically active agents, for example, collagen, gelatin, growth factor, antibiotics, etc. Dissolving all components in solvents presents potential health or cytotoxicity concern due to the solvent residues. Certain reactive bioactive components, such as bioglass can degrade polymer during thermal processing.

Biodegradable dental membranes that can be degraded over time matching the healing of the tissue and avoid the second surgery presenting advantages over non-degradable counterparts, such as titanium or polytetrafluoroethylene. Collagen has been used in biodegradable dental membranes primary due to its bioactivity in facilizing proliferation and differentiation of specialized cells. However, its animal origination, potential immunogenic reaction, and rapid degradation has stimulated the need for safe and degradation tunable alternatives, for example, synthetic polymers, for dental membranes.

Among these synthetic biodegradable polymers are PLLA, PDLA, PDLLA, PLGA, which normally take more than 1 year to degrade. Poly(caprolactone) and its copolymer with L-lactide represent flexible polymers with even longer degradation time. PDO has been characterized as a fast degradation polymer and normally takes less than 6 months to degrade.

However, materials made from these materials are pre-fabricated regardless of applications and actual difference of individual patient. Surgeons usually have to manipulate the materials, e.g. membranes, shapes or sizes to better fit the site. Therefore, a biodegradable material capable of chair-side handling is desired, which could provide additional handling convenience.

US20090286886A1 discloses a biocompatible and water soluble polymer composition based upon poly(alkylene)-poly(ethylene glycol) block copolymer with malleability for use in medicine, dentistry or surgery. The water soluble polymers immediately absorb water or body fluids causing dissolution in water. They can be formulated to stay at the site for a few hours to several days. For dental membrane application, the guided tissue regeneration, usually requires the membrane stay to maintain the function for at least 4-6 weeks. The guided bone regeneration usually requires the membrane stay 4-6 months. These water soluble polymers fall short to maintain shape or functions for such a long period. Therefore, it is an object of this invention to provide composition having chair-side handling properties comprises biodegradable synthetic polymer(s) which can degrade through hydrolysis within the body to fit the tissue healing/growth rate. U.S. Pat. No. 7,157,140B1 disclosed malleable thermoplastic composites consisting of non-degradable polymer(s) such as polyethylene and high specific gravity particulate material. U.S. Pat. No. 8,840,913B2 describes implantable and malleable medical materials comprising mineral particles, insoluble collagen fibers and a gel-forming polysaccharide component and/or another added gel-former. U.S. Pat. No. 8,979,536B2 discloses a protected hardenable dental article with stretchable multi-layer polymeric film having at least two dissimilar polymers in separate layers. These non-degradable polymers provided protection to the malleable and curable dental composites. WO2019/157583 A1 discloses a triple layered alloplastic graft consisting of barrier membrane or mesh being a polymer or composite manufactured by 3D printing or electrospinning.

The compositions used in dental membranes, wound healing patches and hemostasis film of the present invention enable convenient chair-side handling inside operation rooms between room and body temperature. The compositions used in dental membranes, wound healing patches and hemostasis film of the present invention also eliminate the need of thermal processing or cytotoxic solvent mixing to introduce biologically active additives.

### SUMMARY OF THE INVENTION

This invention is directed to a dental membrane comprising a composition, wherein the composition comprises at least one biodegradable polymer and optionally at least one bioactive additive;
wherein the composition at a temperature of 20° C. to 45° C. has:
   a) a storage modulus between 1000 Pa and 100 MPa;
   b) a viscosity between 1000 Pa·s and 1 million Pa·s; and
   c) a ratio of loss modulus over storage modulus tan δ between 1 and 30.

In another aspect, disclosed is a wound healing patch comprising a composition, wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

In another aspect, disclosed is a hemostasis film comprising a composition wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

In another aspect, disclosed is a method of identifying a material as having chair-side handling properties, comprising the steps:
a) placing the material on an oscillatory rheometer or a dynamic mechanical analyzer;
b) performing a temperature sweep method or a temperature ramp method with a frequency in the range of 0.01-100 Hz, preferably in the range of 0.1-10 Hz, more preferably in a range of 0.5-5 Hz, and most preferably at 1 Hz, and a shear strain between 0.01% to 5%, preferably in the range of 0.1% to 2%, more preferably in the range of 0.1% to 1%, and most preferably at 0.5%;
c) heating or cooling the material with a rate of 0.5-10° C./step, and more preferably at 5° C./step or 0.5-10° C./minute, and more preferably at 5° C./min, wherein the material is considered to have chair-side handling properties, if the material at a temperature of 20° C. to 45° C. has:
i) a storage modulus between 1000 Pa and 100 MPa;
ii) a viscosity between 1000 Pa·s and 1 million Pa·s; and
iii) a ratio of loss modulus over storage modulus tan δ between 1 and 30.

Additional advantages will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The above and other objects, features and advantages of the present invention will be more clearly understood from the following detailed description taken in conjunction with the accompanying drawing, in which:

FIG. 1 depicts the rheological behavior of PDO-co-PCL (1,4-Dioxane-2-one and &-Caprolactone molar ratio 20:80).

FIG. 2 depicts the pH value change of the PDO-co-PCL (20:80) in 1×PBS solution at 37° C. without buffer solution change, which represents an accelerated degradation manner.

FIG. 3 depicts the dynamic rheological properties of PDLLA-b-PEG-b-PDLLA with collagen-like protein at percentage by weight of 10%, 20%, 30%, 40%, and 50%.

FIG. 4 depicts the dynamic rheological properties of PDLLA-b-PEG-b-PDLLA with 10 wt % and 20 wt % bioglass, respectively.

FIG. 5 depicts the dynamic rheological properties of PDLLA-b-PEG-b-PDLLA with 10 wt % hyaluronic acid.

FIG. 6 depicts the dynamic rheological properties of PDLLA-b-PEG-b-PDLLA with 10 wt % hhydroxyapatite.

FIG. 7 depicts the dynamic rheological properties of poly(D,L-lactic acid-) (PDLLA) with and without low molecular poly(ethylene glycol) (PEG, Mn=400).

FIG. 8 depicts the dynamic rheological properties of random RG copolymer of poly(D,L-lactic acid-co-glycolic acid) with and without low molecular poly(ethylene glycol) methyl ether (mPEG).

FIG. 9-10 depict the dynamic rheological properties of PEG based polymers with different PEG ratios.

FIG. 11 depicts the dynamic rheological properties of random copolymer poly(lactide-co-caprolactone) with lactide/caprolactone molar ratio of 1:1.

FIG. 12 depicts the dynamic rheological properties of random copolymer of poly(D,L-lactic acid) with inherent viscosity of 0.1 dL/g.

FIG. 13 depicts the dynamic rheological properties of random copolymer of poly(D,L-lactic acid-co-glycolic acid) with lactide/glycolide molar ratio of 1:1.

### DEFINITION OF TERMS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s)”, “include(s)”, “having”, “has”, “can”, “contain(s)”, and variants thereof, as used herein, are intended to be open-ended transitional phrases, terms, or words that do not preclude the possibility of additional acts or structures. The singular forms “a”, “an” and “the” include plural references unless the context clearly dictates otherwise. The present disclosure also contemplates other embodiments “comprising”, “consisting of” and “consisting essentially of”, the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4”. The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “wt. %” means weight percent.

The term “w/w” means weight per weight.

The term “GTR” means guided tissue regeneration. GTR is a technique used to repair periodontal defects so that a tooth, or set of teeth, has more support and stability.

The term “GBR” means guided bone regeneration. GBR is a reconstructive procedure of alveolar ridge using membranes. This procedure is indicated when there is no sufficient bone for implantation, or in the case of optimal implant installation for esthetic or functional needs.

The term “storage modulus” relates to a material's ability to store energy elastically in an oscillatory experiment.

The term “loss modulus” represents the viscous part or the amount of energy dissipated in the sample through heat.

The term “tan δ” is a ratio between loss modulus (G″) and storage modulus (G′) or G″/G′, which is a measure of the relative degree of energy dissipation or damping of the material.

The term “temperature sweep” means temperature change rate in terms of steps, for example, 5° C. per step means temperature change at every 5° C. interval.

The term “temperature ramp” means temperature change rate in term of time, for example, 5° C. per second, or 5° C. per minute, or 5° C. per hour.

For the purposes of the present invention, the term “biodegradable polymers” refers to polymers that dissolve or degrade in vitro or in vivo within a period of time that is acceptable in a particular therapeutic situation. Such dissolved or degraded product may include a smaller chemical species.

Degradation can result, for example, by enzymatic, chemical and/or physical processes.

Biodegradation takes typically less than five years and usually less than one year after exposure to a physiological pH and temperature, such as a pH ranging from 6 to 9 and a temperature ranging from 22° C. to 40° C.

Suitable biodegradable polymers of the invention include without limitation poly(lactide), a poly(glycolide), a poly(lactide-co-glycolide), a poly(caprolactone), a poly(orthoester), a poly(phosphazene), a poly(hydroxybutyrate) a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations thereof. The biodegradable polymer can comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In some aspects, the biodegradable polymer comprises one or more lactide residues. The polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers. Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available.

Homopolymers of lactide or glycolide are also commercially available. 201600168 Page 12/52 When poly(lactide-co-glycolide), poly(lactide), or poly(glycolide) is used, the amount of lactide and glycolide in the polymer can vary. For example, the biodegradable polymer can contain 0 to 100 mole %, 40 to 100 mole %, 50 to 100 mole %, 60 to 100 mole %, 70 to 100 mole %, or 80 to 100 mole % lactide and from 0 to 100 mole %, 0 to 60 mole %, 10 to 40 mole %, 20 to 40 mole %, or 30 to 40 mole % glycolide, wherein the amount of lactide and glycolide is 100 mole %. In a further aspect, the biodegradable polymer can be poly(lactide), 95:5 poly(lactide-co-glycolide) 85:15 poly(lactide-co-glycolide), 75:25 poly(lactide-co-glycolide), 65:35 poly(lactide-coglycolide).

For the purposes of the present invention, the term “combination products” refers to therapeutic and diagnostic products that combine drugs, devices, and/or biological products.

The term “biological tissues” includes, but is not limited to, human soft tissues, skin, subcutaneous layer, mucous membranes, cartilage, ligaments, tendons, muscle tissues, blood vessels, human organs, cardiac muscle tissues, heart valves, nervous tissues, pericardium, pleurae, and peritoneum.

Suitable bioactive additives include, but are not limited to cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.

In another embodiment, thermal processing incudes, but is not limited to single screw extrusion, twin screw extrusion, compression molding, thermal forming, additive manufacturing or 3D printing, and injection molding.

The typical degradation profiles of compositions with chair-side handling properties, or a mixture thereof can be at least two weeks, at least one month, at least 3 months, at least 6 months, at least 9 months, at least 12 months, at least 18 months, or at least 24 months.

Inherent Viscosity (IV) is measured at 25° C. by an automatic viscometer (RPV-1 (2) RSS, PSL Rheoteck) following ASTM D 2857 Test Method for Standard Practice for Dilute Solution Viscosity of Polymers. The polymer was dissolved in chloroform to make 0.1% solution.

Chair-side handling property refers to complex characteristics of compositions or medical devices comprising these compositions so that the compositions inter alia can be deformed under pressure, for example being compressed, or hammered, or rolled, or reshaped without breaking at 20 to 45° C. The chair-side handling property in general relates to a combination of specific viscoelastic properties. The present invention develops a method using oscillatory rheometer or dynamic mechanical analyzer which can distinguish the polymer's viscous and elastic contribution. When the viscous contribution dominates over the elastic contribution, namely the ratio of viscous/elastic portion is greater than 1, the polymer is in a non-recoverable phase and easy to change shape and maintain the new shape. Therefore, chair-side handling property is defined to meet the following conditions in a temperature range of 20-45° C.: 1). a ratio between loss modulus (G″) and storage modulus (G′), also the loss factor (tan δ), greater than 1 and less than 30; 2). storage modulus value between 1000 Pa and 100 MPa and 3). viscosity between 1000-1 million Pa·s. Below this viscosity the polymer or composition loses strength and become flowable, and even sticky to decrease handling property. This method is particularly useful to determining and screening materials, and to determine if whether they can be reshaped manually. Medical implants made with this type material facilitates chair-side good handling property in operation room. Therefore, for the purpose of this present invention, this material has chair-side handling property in a temperature range between room and body temperatures. Instruments used in this method include a rotational rheometer or a dynamic mechanical analyzer.

In a typical testing, for example, by a rotational rheometer (AR2000ex, TA Instruments). 1 g of the materials was loaded on the plate. Lowering the top Peltier plate until a gap of 1.05 mm was reached. After trimming the polymer edge, the Peltier plate was further lowered until a gap of 1.0 mm was reached. A temperature sweep method with 1 Hz and 0.5% shear strain at a rate of 5° C./step was used. The testing can be performed in a cooling mode from polymer melt to room temperature or in a heating mode from ambient or sub-ambient temperature.

In one embodiment, the composition may have a range of PDO/PCL between 10:90 and 30:70. In one embodiment, the biodegradable polymer of the composition may have a molecular weight range from 10 k to 150 k g/mol, preferably in a range from 20 k to 120k g/mol, and more preferably within a range from preferably 30 k to 100 k g/mol.

Item 1 is a dental membrane comprising a composition, wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

Item 2 is the dental membrane of item 1, wherein the biodegradable polymer is selected from a polydioxanone, a polycaprolactone, a poly(orthoester), poly(D,L-lactic acid), polyglycolide, polyethylene glycol (PEG), and a copolymer, a terpolymer or a mixture thereof.

Item 3 is the dental membrane according to any of items 1 to 2, wherein the bioactive additive is cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.

Item 4 is a wound healing patch comprising a composition, wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

Item 5 is the wound healing patch of item 4, wherein the biodegradable polymer is selected from a polydioxanone, a polycaprolactone, a poly(orthoester), poly(D,L-lactic acid), polyglycolide, polyethylene glycol (PEG) and a copolymer, a terpolymer or a mixture thereof.

Item 6 is the wound healing patch according to any of items 4 to 5, wherein the bioactive additive is cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.

Item 7 is a hemostasis film comprising a composition wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

Item 8 is the hemostasis film of item 7, wherein the biodegradable polymer is selected from a polydioxanone, a polycaprolactone, a poly(orthoester), poly(D,L-lactic acid), polyglycolide, polyethylene glycol (PEG), and a copolymer, a terpolymer or a mixture thereof.

Item 9 is the hemostasis film according to any of items 7 to 8, wherein the bioactive additive is cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.

Item 10 is a method of identifying a material as having chair-side handling properties, comprising the steps:
a) placing the material on an oscillatory rheometer or a dynamic mechanical analyzer;
b) performing a temperature sweep method or a temperature ramp method with a frequency in the range of 0.01-100 Hz, preferably in the range of 0.1-10 Hz, more preferably in a range of 0.5-5 Hz, and most preferably at 1 Hz, and a shear strain between 0.01% to 5%, preferably in the range of 0.1% to 2%, more preferably in the range of 0.1% to 1%, and most preferably at 0.5%;
c) heating or cooling the material with a rate of 0.5-10° C./step, and more preferably at 5° C./step or 0.5-10° C./minute, and more preferably at 5° C./min, wherein the material is considered to have chair-side handling properties, if the material at a temperature of 20° C. to 45° C. has:
   i) a storage modulus between 1000 Pa and 100 MPa;
   ii) a viscosity between 1000 Pa·s and 1 million Pa·s; and
   iii) a ratio of loss modulus over storage modulus tan δ between 1 and 30.

Item 11 is the method according to claim 10, wherein the material is a dental membrane, wound healing patch or hemostasis film.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature if no temperature is indicated, and pressure is at or near atmospheric pressure.

In general, the materials of this invention can be made by processes which include processes analogous to those known in the chemical arts, particularly in light of the description contained therein.

### Preparation of Example 1

### Synthesis of Polydioxanone-Co-Polycaprolactone

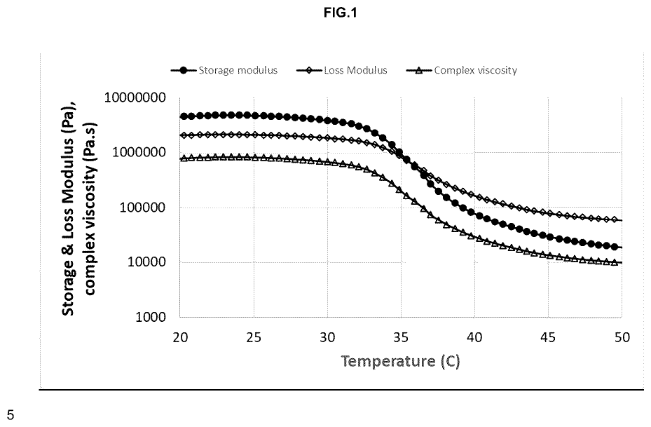

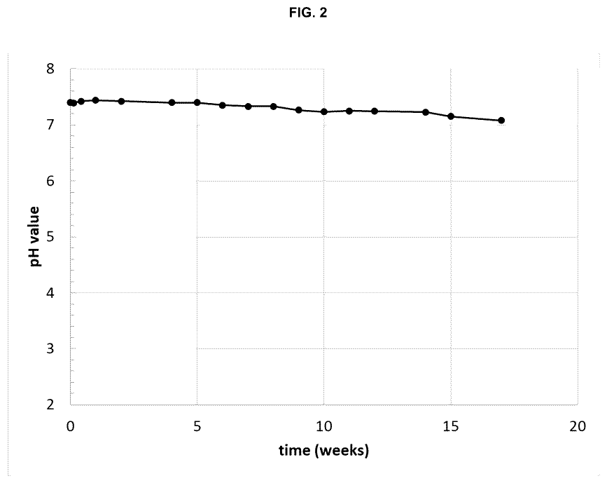

1,4-Dioxane-2-one (DO) was melted at 40° C. for 5 hours in a digital incubator. The melted DO (8 mL) was transferred to a reactor. The DO and reactor were dried under N₂ gas overnight. Stannous octoate (20 μL) solution was prepared with toluene (10 mL). ε-Caprolactone (CL; 37 mL), 1,4-butanediol (46.8 μL) and stannous octoate solution (1 mL) were added to the reactor. Reactor was heated up to 45° C. to melt DO and prepare homogenous solution. The polymerization was carried out at 140° C. for 28 hours. After polymerization was completed, chloroform was added to make polymer solution. Polymer was precipitated in cold methanol and was dried in vacuum oven for 3 days. ¹H NMR confirmed the polymer chemical structure, the monomer composition of DO (18.9%) and CL (81.1%). Molecular weight (Mw: 109 k g/mol, Mn: 74 k g/mol) and molecular weight distribution (PDI: 1.471) were determined by GPC. Measurement results are shown in FIG. 1 indicating chair-side handling properties. In vitro degradation results are shown in FIG. 2.

A series of PDO-co-PCL with various DO/CL were synthesized. Measured values are listed in Table 1.

### Preparation of Example 2

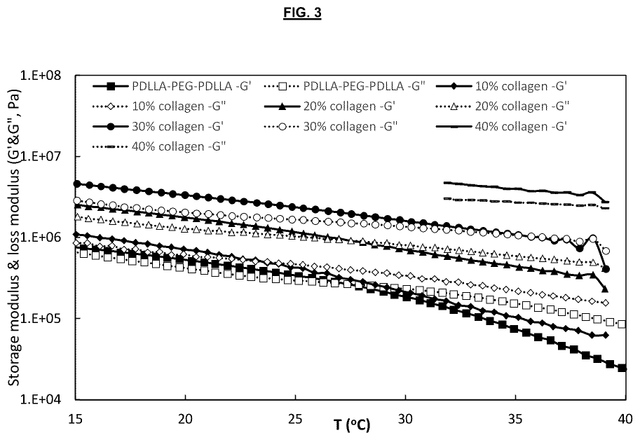

Preparation of PDLLA-b-PEG-b-PDLLA block copolymer with collagen-like protein PDLLA-b-PEG-b-PDLLA block copolymer 100DL PEG 6000 (30 wt % PEG) (Evonik Corporation) and collagen-like protein (Evonik Corporation) were weighed out and combined in various ratios (100/0, 97/3, 90/10, 80/20, 70/30, 60/40, and 50/50). To simulate chair-side handling process, the weighed collagen-like protein was manually kneaded into PDLLA-PEG-PDLLA block copolymer for 2 minutes at room temperature. 1 g of PDLLA-PEG-PDLLA block copolymer with collagen-like protein at each ratio was prepared. Alternatively, the mixture of collagen-like protein and PDLLA-PEG-PDLLA block copolymer could be mixed in a container by high speed mixer at room temperature. The obtained compositions showed chair-side handling properties and were active compositions. Measurement results are shown in FIG. 3.

### Preparation of Example 3

### Preparation of PDLLA-b-PEG-b-PDLLA Block Copolymer with Bioglass

PDLLA-b-PEG-b-PDLLA block copolymer 100DL PEG 6000 (30 wt % PEG) (Evonik Corporation) and bioglass (Schott AG) were weighed out and combined at ratios of 90:10 and 80:20, respectively. The weighed bioglass were manually kneaded into PDLLA-b-PEG-b-PDLLA block copolymer for 2 minutes at room temperature. 1 g of PDLLA-b-PEG-b-PDLLA block copolymer with bioglass at each ratio was prepared. Alternatively, the mixture of bioglass and PDLLA-b-PEG-b-PDLLA block copolymer could be mixed in a container by high speed mixer at room temperature.

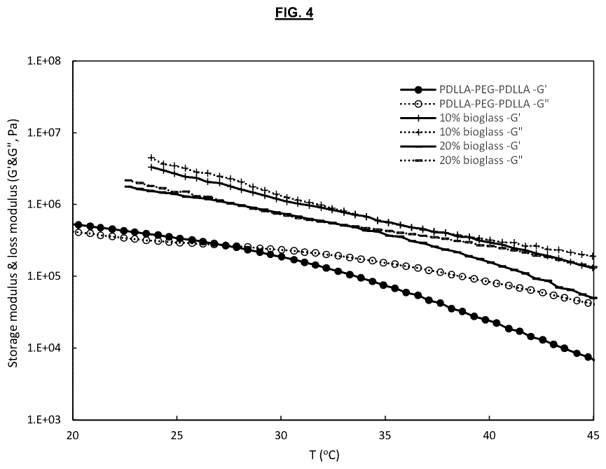

No polymer degradation was observed. The bioglass fillers were uniformly dispersed in the polymer. Measurement results are shown in FIG. 4. The obtained compositions showed chair-side handling properties and were active compositions.

### Preparation of Example 4

### Preparation of PDLLA-b-PEG-b-PDLLA block copolymer with hyaluronic acid

PDLLA-b-PEG-b-PDLLA block copolymer 100DL PEG 6000 (30 wt % PEG) (Evonik Corporation) and hyaluronic acid (Evonik Corporation) at a ratio of 90:10 by weight were manually kneaded for 2 minutes at room temperature. Alternatively, the mixture of hyaluronic acid and PDLLA-b-PEG-b-PDLLA block copolymer could be mixed in a container by high speed mixer at room temperature.

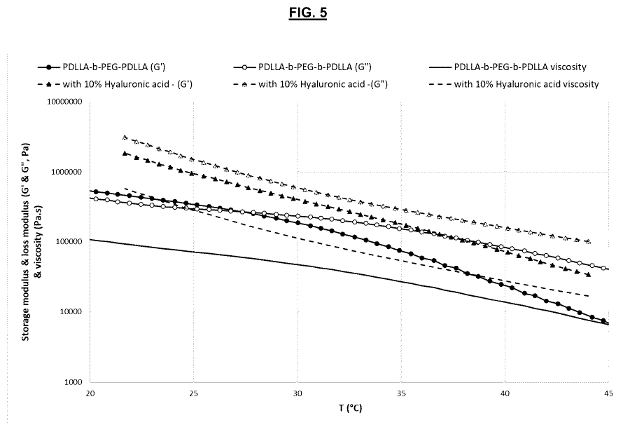

Measurement results are shown in FIG. 5. The obtained compositions showed chair-side handling properties and were active compositions.

### Preparation of Example 5

### Preparation of PDLLA-b-PEG-b-PDLLA Block Copolymer with Hydroxyapatite

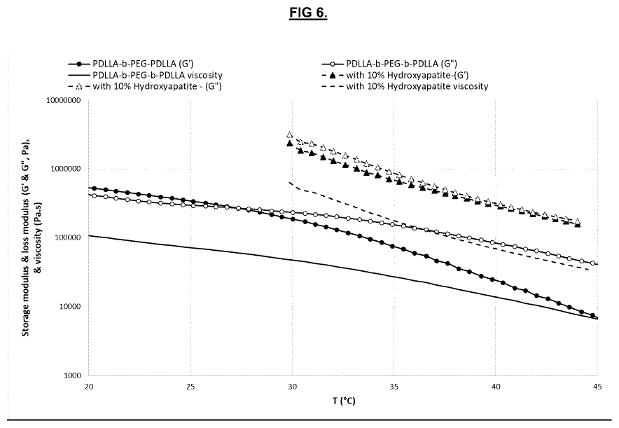

PDLLA-b-PEG-b-PDLLA block copolymer 100DL PEG 6000 (30 wt % PEG) (Evonik Corporation) and hydroxyapatite (Plasma Group Science) at a weight ratio of 90:10 and 80:20, respectively, were manually kneaded for 2 minutes at room temperature. Alternatively, the mixture of hydroxyapatite and PDLLA-b-PEG-b-PDLLA block copolymer could be mixed in a container by high speed mixer at room temperature. Measurement results are shown in FIG. 6. The obtained compositions showed chair-side handling properties and were active compositions.

### Preparation of Example 6

### Preparation of Material from Poly(Ethylene Glycol) and Poly(D,L-Lactide)

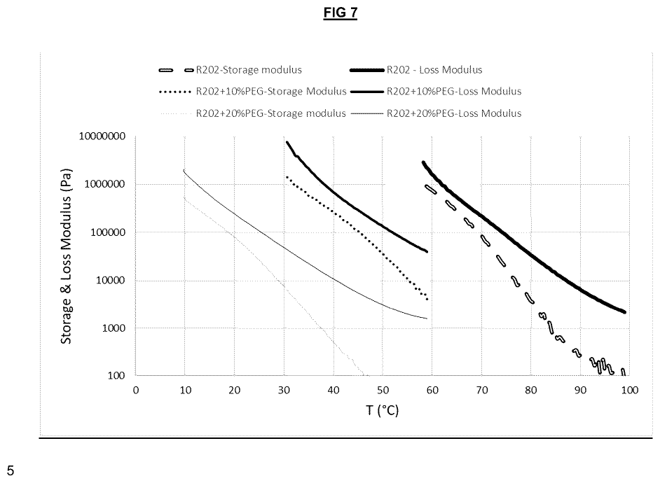

Poly(D,L-lactide) (RESOMER® R 202 H, Evonik Corporation) with poly(ethylene glycol) (Mn=400, commercially available at Millipore Sigma) at different ratios (10%, 20%, 30%, 40%, and 50%) were thermally compounded by a HAAKE MinLab twin screw microcompounder. The 3 heating zones for the microcompounder were set at 100° C., respectively. The PDLLA with PEG were fed into the microcompounder and recirculated for 3 minutes and discharged to a sample collecting tray. The extrudates were cooled down to room temperature for overnight. Then the characteristics were evaluated by rotational rheometer. The polymers with more than 30% PEG were flowable and sticky and therefore not characterized. The results are shown in FIG. 7. The obtained compositions showed chair-side handling properties and were active compositions. Compositions with poly(D,L-lactide) only did not show chair-side handling properties.

### Preparation of Example 7

### Preparation of Polymer from Poly(Ethylene Glycol) Methyl Ether and Poly(D,L-Lactide-Co-Glycolide)

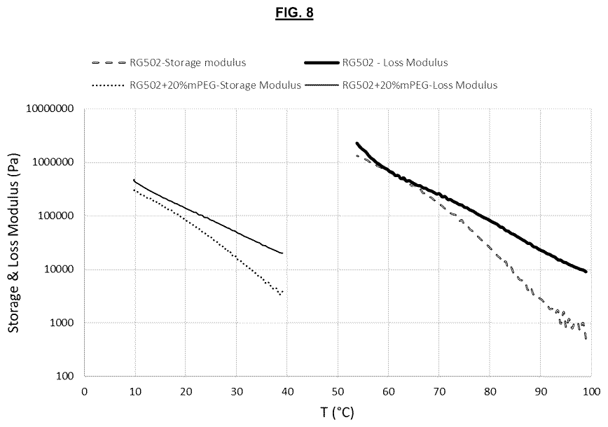

Poly(D,L-lactide-co-glycolide) (RESOMER® RG 502, Evonik Corporation) with poly(ethylene glycol) methyl ether (Mn=550, commercially available at Millipore Sigma) at different ratios (20%, 30%, and 40%) were mixed in dichloromethane and stirred at room temperature until all polymers completely dissolved. The clear solutions were then casted on glass petri dish and allowed the solvent to evaporate. The dried samples were then evaluated on rheometer. The results are shown in FIG. 8. The obtained compositions showed chair-side handling properties. Compositions with poly(D,L-lactide-co-glycolide) only did not show chair-side handling properties.

### Example 8

### Side-Chair Handling Properties Characterization and Screen Method Setup

Characteristics (storage modulus, viscosity, and ratio of loss modulus over storage modulus) of the materials was evaluated by a rotational rheometer (AR 2000ex, TA Instruments). 1 g of the materials was loaded onto the plate. Lowering the top Peltier plate until a gap of 1.05 mm was reached. After trimming the polymer edge, the Peltier plate was further lowered until a gap of 1.0 mm was reached. A temperature sweep method with 1 Hz and 0.5% shear strain at a rate of 5° C./step was used. Storage modulus, viscosity, and ratio of loss modulus over storage modulus was measured at temperatures of 20 to 45° C.

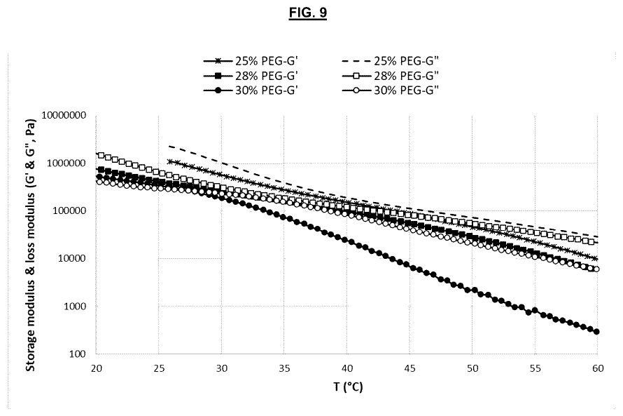

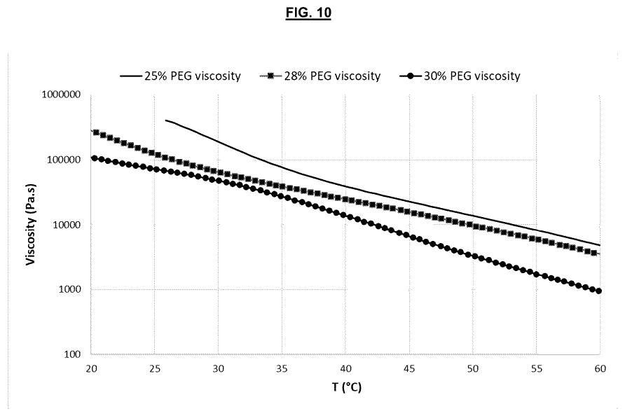

A number of biodegradable polymers were screened: poly(ethylene glycol) contained block copolymers: poly(D,L-lactide)-b-PEG, with 15 wt %, 22 wt %, 25 wt %, 28 wt % PEG, respectively; poly(D,L-lactide)-b-PEG-b-poly(D,L-lactide) with 30 wt % PEG; and PDLLA-b-PEG-b-PDLLA (RESOMER®: RP t 7046, Evonik product). The detail PDLLA and PEG segment molecular weight and side-chair handling properties were listed in Table 2. Results are shown in FIG. 9-10.

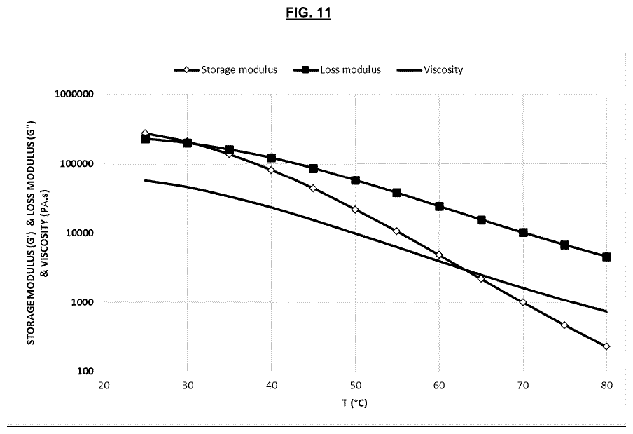

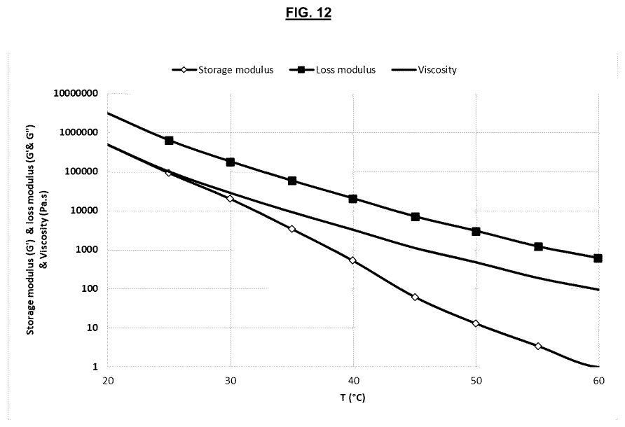

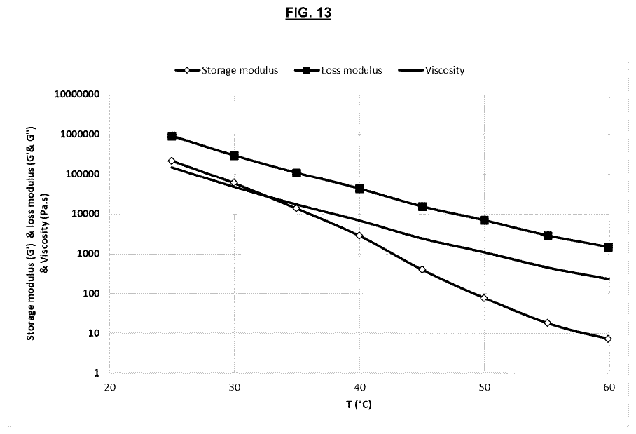

The following polymers (Evonik products) exhibited side-chair handling properties: random copolymer poly(lactide-co-caprolactone) with lactide/caprolactone molar ratio of 1:1 (FIG. 11), random copolymer of poly(D,L-lactic acid) with inherent viscosity of 0.1 dL/g (FIG. 12), random copolymer of poly(D,L-lactic acid-co-glycolic acid) with lactide/glycolide molar ratio of 1:1 (FIG. 13).

### Testing

During the in vitro degradation, phosphate-buffered saline (PBS) 1× solution (Fisher BioReagents, Pittsburgh, PA) with pH 7.4±0.1 was used in this study. This PBS solution contains 11.9 mM phosphates, 137 mM sodium chloride, and 2.7 mM potassium chloride. The PDO-co-PCL specimens were placed in the PBS solution at 37° C. in an incubator. Two types of in vitro degradation studies performed. In one study, the PBS buffer solution was replaced weekly. In another accelerated study, the specimens were immersed in the PBS buffer solution without changing it all the time. Upon reaching designated degradation time, the specimens were allowed to cool down to room temperature prior to pH value check.

### Results

FIG. 1 depicts the dynamic rheological properties of polydioxanone-co-polycaprolactone (20:80).

The loss modulus (G″) is greater than storage modulus (G′) when temperature is higher than 35° C. The storage modulus is less than 10 MPa in a temperature range between 2° and 50° C. Complex viscosity is higher than 1000 Pa·s in the investigated temperature range.

FIG. 2 depicts the pH value change of polydioxanone-co-polycaprolactone (20:80) in an accelerated degradation manner. The polymer specimens were immersed in 1×PBS buffer solution at 37° C. without the change of buffer solution. The possible degradation products remain in the solution which includes possible acid. Within the investigated four months, there was a minimal change in pH value.

FIG. 3 depicts the dynamic rheological properties of PDLLA-b-PEG-b-PDLLA with collagen-like protein at percentage by weight of 10%, 20%, 30%, 40%, and 50%. The loss modulus (G″) is greater than storage modulus (G′) within the investigated temperature range 20-45° C. for the composition with 10%, 20%, 30%, 40%, 50% collagen-like protein. In all the composition, the storage modulus is less than 10 MPa in a temperature range between 2° and 45° C. These materials show chair-side handling properties.

FIG. 4 depicts the dynamic rheological properties of PDLLA-b-PEG-b-PDLLA with 10 wt % and 20 wt % bioglass, respectively. The loss modulus (G″) is close to or greater than storage modulus (G′) within the investigated temperature range for both compositions. The loss modulus (G″) is very closed to storage modulus (G′) within the temperature range 27-33° C. for 10 wt % bioglass, meanwhile the loss modulus (G″) is closed to storage modulus (G′) within the temperature range 33-37° C. for 20 wt % bioglass. In all the composition, the storage modulus is less than 10 MPa in the investigated temperature range. These compositions facilitate good chair-side handling properties.

There is no polymer degradation with the presence of bioglass fillers.

FIG. 5 depicts the dynamic rheological properties of PDLLA-b-PEG-b-PDLLA with 10 wt % hyaluronic acid. The loss modulus (G″) is greater than storage modulus (G′) within the investigated temperature range for both compositions. The storage modulus is less than 10 MPa in the investigated temperature range. These compositions facilitate good chair-side handling properties.

FIG. 6 depicts the dynamic rheological properties of PDLLA-b-PEG-b-PDLLA with 10 wt % hhydroxyapatite. The loss modulus (G″) is greater than storage modulus (G′) within the temperature range 35-40° C. for 10 wt % hydroxyapatite. The storage modulus is less than 10 MPa in the investigated temperature range. These compositions facilitate good chair-side handling properties.

FIG. 7 depicts the dynamic rheological properties of poly(D,L-lactic acid-) (PDLLA) with and without low molecular poly(ethylene glycol) (PEG, Mn=400). The PDLLA polymer has loss modulus (G″) value which is greater than storage modulus (G′) in the investigated temperature 60-100° C. The PDLLA polymer with 10% PEG has loss modulus greater than its storage modulus in the investigated temperature range 30-60° C. The storage modulus is less than 1 MPa in this temperature range. The PDLLA polymer with 20% PEG has loss modulus greater than its storage modulus in the investigated temperature range 10-60° C.

FIG. 8. depicts the dynamic rheological properties of random RG copolymer of poly(D,L-lactic acid-co-glycolic acid) with and without low molecular poly(ethylene glycol) methyl ether (mPEG). The RG polymer has loss modulus (G″) value which is greater than storage modulus (G′) when the temperature is higher than 60° C. This RG polymer does not have chair-side handling property when temperature is below 60° C. The RG polymer with 20% mPEG has loss modulus greater than its storage modulus in the investigated temperature range 10-40° C. The storage modulus is less than 1 MPa in a temperature range between 10 and 40° C. The RG polymer with 20% mPEG has chair-side handling property at body temperature (37° C.).

FIG. 9-10. depict the dynamic rheological properties of PEG based polymers with different PEG ratios. The loss modulus (G″) is greater than storage modulus (G′) within the temperature range 28-45° C. for the PDLLA-PEG polymer with 25 wt %, 28 wt % and 30 wt % PEG. In all the composition, the storage modulus is less than 100 MPa in a temperature range between 28 and 45° C. viscosity is above 1000 Pa·s in a temperature range between 2° and 45° C. These materials show chair-side handling properties.

FIG. 11 depicts the dynamic rheological properties of random copolymer poly(lactide-co-caprolactone) with lactide/caprolactone molar ratio of 1:1. The inherent viscosity (IV) is 1.5 dL/g.

The loss modulus (G″) is greater than storage modulus (G′) when temperature is higher than 35° C. The storage modulus is less than 1 MPa in a temperature range between 25 and 80° C. The polymer shows chairs-side handling properties at body temperature (37° C.).

FIG. 12. depicts the dynamic rheological properties of random copolymer of poly(D,L-lactic acid) with inherent viscosity of 0.1 dL/g. The loss modulus (G″) is greater than storage modulus (G′) in a temperature range 25-60° C. The storage modulus is less than 1 MPa in a temperature range between 25 and 60° C. This low molecular weight polymer shows chair-side handling properties at body temperature (37° C.).

FIG. 13. depicts the dynamic rheological properties of random copolymer of poly(D,L-lactic acid-co-glycolic acid) with lactide/glycolide molar ratio of 1:1. The inherent viscosity (IV) is 0.1 dL/g. The loss modulus (G″) is greater than storage modulus (G′) in a temperature range 25-60° C. The storage modulus is less than 1 MPa in a temperature range between 25 and 60° C. This low molecular weight polymer shows chair-side handling properties at body temperature (37° C.). The polymer with IV value greater than 0.2 dL/g loses chairs-side handling properties and becomes rigid and brittle.

Table 1 depicts synthesized polydioxanone-co-polycaprolactone polymers with various ratios between PDO and PCL and with various molecular weight. The polymers depend on the ratios of PDO and PCL and the molecular weight, they can be liquid at room temperature; flexible at room temperature and show chair-side handling properties when temperature is higher than 35° C.; rigid powders at room temperature.

TABLE 1

| Planed | GPC | | NMR | | |
|---|---|---|---|---|---|
| composition | Actual | Actual | Actual | | Chair-side handling |
| (PDO/PCL) | Mn (k) | PDI | DX | CL | property |
| 35/65 | 21.9 | 1.47 | 35.6 | 64.4 | No - liquid |
| 20/80 | 30.8 | 1.53 | 18.5 | 81.5 | Yes |
| 20/80 | 74.0 | 1.47 | 18.9 | 81.1 | Yes |
| 80/20 | 24.1 | 1.65 | 81.5 | 18.5 | No - powder |
| 30/70 | 20.7 | 1.54 | 31.7 | 68.3 | No - liquid |

Table 2 depicts polyethylene glycol based copolymer with polylactide at different ratio and PEG segment ratios.

TABLE 2

| | | | | Chair-side |
|---|---|---|---|---|
| | PEG | PDLLA | PEG | handling |
| Composition | (wt %) | (kDa) | (kDa) | property |
| PDLLA-b-PEG | 15 | 28 | 5 | No |
| PDLLA-b-PEG | 22 | 18 | 5 | No |
| PDLLA-b-PEG | 25 | 15 | 5 | Yes |
| PDLLA-b-PEG | 28 | 13 | 5 | Yes |
| PDLLA-b-PEG-b-PDLLA | 30 | 14 | 6 | Yes |
| PDLLA-b-PEG-b-PDLLA | 4 | 70% L/ | 9 | No |
| (RESOMER ® LRP t7046) | | 30% DL | | |

What is claimed is:

1. A dental membrane comprising a composition, wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

2. The dental membrane of claim 1, wherein the biodegradable polymer is selected from a polydioxanone, a polycaprolactone, a poly(orthoester), polyethylene glycol (PEG), and a copolymer, a terpolymer or a mixture thereof.

3. The dental membrane according to claim 1, wherein the bioactive additive is cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.

4. A wound healing patch comprising a composition, wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

5. The wound healing patch of claim 4, wherein the biodegradable polymer is selected from a polydioxanone, a polycaprolactone, a poly(orthoester), polyethylene glycol (PEG) and a copolymer, a terpolymer or a mixture thereof.

6. The wound healing patch according to claim 4, wherein the bioactive additive is cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.

7. A hemostasis film comprising a composition wherein the composition comprises at least one biodegradable polymer and optionally at least one bioactive additive; wherein the composition at a temperature of 20° C. to 45° C. has:
a) a storage modulus between 1000 Pa and 100 MPa;
b) a viscosity between 1000 Pa·s and 1 million Pa·s; and
c) a ratio of loss modulus over storage modulus tan δ between 1 and 30.

8. The hemostasis film of claim 7, wherein the biodegradable polymer is selected from a polydioxanone, a polycaprolactone, a poly(orthoester), polyethylene glycol (PEG), and a copolymer, a terpolymer or a mixture thereof.

9. The hemostasis film according to claim 7, wherein the bioactive additive is cellulose microcrystal, chitin whisker, collagen-like protein, crosslinked collagen-like protein, silk, hydroxyapatite, fluor-hydroxyapatite, fluorapatite, calcium carbonate, calcium phosphate, β-tricalcium phosphate, magnesium, magnesium phosphate, bioglass, active pharmaceutical ingredient, or a mixture thereof.
